(19) United States
(12) Patent Application Publication
(10) Pub. No.: US 2009/0286876 A1
(43) Pub. Date: Nov. 19, 2009

(54) NEUROPROTECTIVE BENZOATE AND BENZAMIDE COMPOUNDS

(75) Inventors: Laurent Lecanu, McLean, VA (US); Janet Greeson, Las Vegas, NV (US); Vassilios Papadopoulos, North Potomac, MD (US)

Correspondence Address: Georgetown University (c/o Sharon Prisco);Office of Technology Commercialization, 3300 Whitehaven Street NW, Harris Building, Suite 1500, Washington, DC, 20007, US

(73) Assignees: Samaritan Pharmaceuticals, Inc.; Georgetown University

(21) Appl. No.: 12/269,559

(22) Filed: Nov. 12, 2008

Related U.S. Application Data
(63) Continuation of application No. 11/292,781, filed on Dec. 2, 2005, now abandoned.
Application No. 11/292,781 is a continuation of application No. PCT/US2004/016036, filed on May 20, 2004.
(60) Provisional application No. 60/474,964, filed on Jun. 2, 2003.
(60) Provisional application No. 60/475,642, filed on Jun. 4, 2003.
(60) Provisional application No. 60/478,648, filed on Aug. 1, 2003.
(60) Provisional application No. 60/566,869, filed on Apr. 30, 2004.

(51) Int. Cl. A61K 31/24; A61K 31/166; A61K 31/164; A61P 25/28; A61P 25/02

(52) U.S. Cl. 514/535; 514/619; 514/626

(57) ABSTRACT

The invention provides a therapeutic method for treating at least one symptom of a neurological disorder or disease such as Alzheimer's disease in a mammal, such as a human, wherein the toxicity of a pathogen of β amyloid peptide and/or glutamate in mammalian cells is implicated and inhibition of the subsequently-induced pathological pathways is desired comprising administering to a mammal in need of such therapy, an effective amount of an N-arylamide or an (N-aminoalkyl)benzamide, including pharmaceutically acceptable salts thereof.

### RELATED APPLICATIONS

The present application is a Continuation of U.S. patent application Ser. No. 11/292,781, filed Dec. 2, 2005, which is a Continuation under 35 U.S.C. 111(a) of International Application No. PCT/US2004/016036 filed May 20, 2004 and published in English as WO 2004/108666 A3 on Dec. 16, 2004, which claimed the benefit of U.S. Provisional Application Ser. Nos. 60/474,964 filed Jun. 2, 2003; 60/475,642 filed Jun. 4, 2003; 60/478,648 filed Aug. 1, 2003; and 60/566,869 filed Apr. 30, 2004; which applications and publication are incorporated by reference in their entirety and made a part hereof.

### BACKGROUND OF THE INVENTION

Alzheimer's disease (AD) is the most common dementia occurring in elderly, affecting about 10% of people above 65 years and 40% above 80 years. The familial AD is the early-onset form of the disease that involves different mutations of the amyloid protein precursor (APP) gene and accounts for no more than 5% of the total AD cases. The late-onset form of the disease, also called sporadic form, accounts for more than 95% of the AD cases and its origins remain elusive. Several risk factors have been identified or are suspected. These include the ε4 allele of the apoE gene, socio-economical situation or previous medical conditions, but a causality relationship of the onset or progression of the disease has not been yet established.

AD is clinically characterized by a progressive and irreversible impairment of cognition processes and memory alteration, and is commonly associated with a non-cognitive symptomotology, including depression (Robert et al., Alzheimer's Disease: from molecular biology to therapy, R. Becker et al., eds., (1996) at 487-493. Alzheimer's disease (AD) neuropathology is histologically characterized by an increase of brain β-amyloid (Aβ) peptide levels accompanied by the formation of senile plaques (Nikaido et al. (1970) Trans Am. Neurol. Assoc. 95:47-50 and the appearance of neurofibrillary tangles (NFT), due to a hyperphosphorylation of the Tau protein (Kosik et al., (1986) PNAS USA 83:4044-8. Aβ is produced by proteolytic cleavage of the β-amyloid precursor protein (β-APP) by the membrane enzymes β- and γ-secretase. Aβ exists either as the most commonly found 40 amino acid length Aβ₁₋₄₀ form on the 42 amino acid Aβ₁₋₄₂ form, reported to be more neurotoxic than Aβ₁₋₄₀. Although understanding of Aβ-medicated neurotoxicity has dramatically increased during the last decade, no Aβ₁₋₄₂ targeting therapeutic strategy has been shown to successfully slow down the progression of the disease. Rather, current therapeutic strategies under investigation for AD include inhibitors of Aβ production, compounds that prevent its oligomerization and fibrillization, anti-inflammatory drugs, inhibitors of cholesterol synthesis, antioxidants, neurorestorative factors and vaccines [Selkoe, D. J. (1999) Nature 399, A23-31; Emilien, G., et al. (2000) Arch. Neurol. 57, 454-459; Klein, W. L. (2002) Neurochem. Internat. 41, 345-52; Helmuth, L. (2002) Science 297(5585), 1260-21.

### SUMMARY OF THE INVENTION

The invention provides a method to treat neuropathologies, such as vascular dementia or hypertension, age-related depression, or mood swings, and Alzheimer's disease, for example, by blocking or inhibiting the ability of glutamate or amyloid, such as Aβ₁₋₄₂, Aβ₁₋₄₀ or Aβ₁₋₄₃, to damage mammalian neurons. Thus, the present invention provides a method for treatment of a mammal threatened or afflicted by a neuropathological condition such as Alzheimer's disease, by administering to said mammal an effective amount of a compound of formula I or formula II:

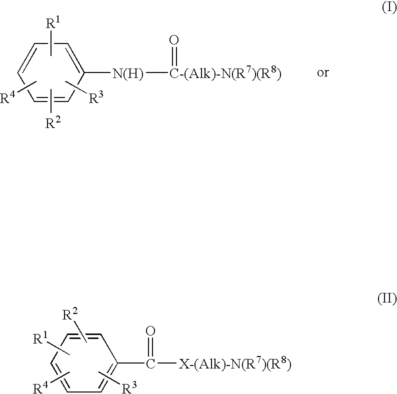

wherein:

a) R¹, R² and R³ are individually H, OH, halo, CN, (C₁-C₆)alkyl, (C₁-C₆)alkoxy, (C₃-C₆)cycloalkyl, (C₃-C₆)cycloalkoxy, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio, thio(C₁-C₆)alkyl-, (C₁-C₆)alkanoyloxy, N(R⁵)(R⁶) or R¹ and R² together are methylenedioxy;

b) R⁴ is hydrogen, (C₁-C₃)alkyl, N(R⁵)(R⁶), (C₁-C₆)alkoxy;

c) R⁵, R⁶, R⁷ and R⁸ are individually, H, (C₁-C₆)alkyl, (C₃-C₆)cycloalkyl, (C₃-C₆)cycloalkyl((C₁-C₆)alkyl), (C₂-C₆)alkenyl, wherein cycloalkyl optionally comprises 1-2, S, nonperoxide O or N(R⁵); aryl, aryl(C₁-C₆)alkyl, aryl(C₂-C₆)alkenyl, heteroaryl, heteroaryl(C₁-C₆)alkyl, or R⁵ and R⁶ or R⁷ and R⁸ together with the N to which they are attached form a 5- or 6-membered heterocyclic or heteroaryl ring, optionally substituted with R¹ and optionally comprising 1-2, S, non-peroxide O or N(R⁵);

d) (Alk) is (C₁-C₆)alkyl, (C₂-C₆)alkenyl, (C₃-C₆)cycloalkyl, (C₃-C₆)cycloalkyl(C₂-C₆)alkyl or [(C₂-C₆)alkyl(C₃-C₆)cycloalkyl[(C₃-C₆)alkyl] optionally substituted by 1-2 S, non-peroxide O or N(R⁵); and

e) X is O or NH;

and the pharmaceutically acceptable salts thereof.

Preferably (Alk) is (C₁-C₄)alkyl, such as —(CH₂)—(CH₂)₂—, —(CH₂)₃— or —(CH₂)₄—.

Preferably, 1 or 2, of R¹, R², R³ or R⁴ is N(R⁵)(R⁶).

Preferably, both of R⁵ and R⁶ is H.

Preferably, one or both of R⁷ and R⁸ are (C₁-C₆)alkyl or (C₃-C₆)cycloalkyl, or one is H and one is (C₁-C₆)alkyl or (C₃-C₆)cycloalkyl.

Preferably, 1 or 2 of R¹, R², R³ or R⁴ is (C₁-C₆)alkoxy.

Preferably, (R⁵)(R⁶)N— is in the para or 4-position in formula (I), preferably two of R¹, R², R³ and R⁴ are not (C₁-C₃)alkyl.

In formula (I), preferably two of R¹, R², R³ and R⁴ are (C₁-C₃)alkyl.

In formula II, preferably R⁷ and R⁸ are both ethyl when one of R¹, R², R³ and R⁴ is 4-amino and three are H.

The invention also provides a pharmaceutical composition comprising a compound of formula I, and/or formula II or a pharmaceutically acceptable salt thereof, in combination with a pharmaceutically acceptable diluent or carrier, and can optionally include stabilizers, preservatives, and absorption control agents.

The invention also provides a pharmaceutical composition such as a unit dosage form, comprising a compound of formula I or II, or a pharmaceutically acceptable salt thereof, in combination with a pharmaceutically acceptable diluent or carrier, which optionally can include one or more anti-AD agents of one or more of the classes of anti-AD agents referenced hereinabove, and can optionally include stabilizers, preservatives, and absorption control agents.

Additionally, the invention provides a therapeutic method for preventing or treating a pathological condition or symptom in a mammal, such as a human, that is associated with AD or the onset of AD, or that is associated with the toxicity of a pathogen such as β-amyloid peptide and/or glutamate toward mammalian neuronal cells, wherein inhibition of said toxicity is desired, or down-modulation of the subsequently induced pathological pathway is desired, comprising administering to a mammal in need of such therapy, an effective amount of a compound of formula I, or a pharmaceutically acceptable salt thereof.

Thus, the invention also provides a therapeutic method to treat a neuropathy that involves glutamate network hyperactivity, such as cerebral ischemia, AIDS-associated dementia, stroke, traumatic brain or spinal cord injury, and the like.

The invention provides a compound of formula I for use in medical therapy (e.g., for use in treating a mammal afflicted or threatened with AD, as well as the use of a compound of formula I or II for the manufacture of a medicament useful for the treatment of at least one AD symptom in a mammal, such as a human, such as an AD patient.

The invention also provides novel compounds of formula I or II, as well as, processes and intermediates disclosed herein that are useful for preparing compounds of formula (I) or salts thereof.

### SUMMARY OF THE FIGURES

FIG. 1 depicts the chemical formula of procaine and of certain procaine derivatives. SP015, SP016 and SP017 were identified by screening a natural compounds database using procaine and procainamide as a substructure.

FIG. 2 (panels A-C) are graphs depicting the effect of Aβ₁₋₄₂ on rat pheochromocytoma PC12 cells cell viability assessed by MTT assay (A) and by measuring the intracellular ATP concentrations (B). The effect of Aβ₁₋₄₂ on the free radical production was assayed using the fluorescent probe 2,7-DCF (C). PC12 cells were exposed to increasing concentrations of Aβ₁₋₄₂ (C=control) and the different parameters were assayed after 24 hours exposure. The statistical analysis was performed using one-way ANOVA followed by Dunnett's test. Mean±SD, n=6. * p<0.05, *** p<0.001 compared to control unless differently specified.

FIG. 3. Protective effect of the non-competitive NMDA antagonist (+)-MK801 against Aβ₁₋₄₂ neurotoxicity. PC12 cells were pre-incubated for 24 hours with increasing concentrations of (+)-MK801 before being exposed for 24 hours to increasing concentrations of Aβ₁₋₄₂. The cell viability was assessed by MTT assay. Control cells (C) wee not exposed neither to (+)-MK801 nor to Aβ₁₋₄₂. The statistical analysis was performed using one-way ANOVA followed by Dunnett's test. Mean±SD, n=6. * p<0.05, *** p<0.001 compared to (+)-MK801 0 μM.

FIG. 4 (panels A-D). Effect of compounds on the Aβ₁₋₄₂-induced free radical production of PC12 cells. P12 cells were pre-incubated for 24 hours with increasing concentrations of procaine (A), lidocaine (B), tetracaine (C) and procainamide (D) before being exposed to increasing concentrations of Aβ₁₋₄₂. The free radical production was measured using the fluorescent probe 2,7-DCF after 24 hours of Aβ₁₋₄₂ exposure. Control cells were exposed neither to pharmacological agents nor to Aβ₁₋₄₂. The statistical analysis was performed using one-way ANOVA followed by Dunnett's test. Mean±SD, n=6, compared to the 0 μM concentration. For clarity concern, the significance stars have not been added to the figure.

FIG. 5. Neuroprotective effect of procaine and SP008 ((4-ethylpiperazinyl-1-yl)-2′,3′,4′-trimethoxybenzoate) against glutamate-induced cell death of PC12 cells. PC12 cells were pre-incubated with increasing concentrations of procaine or SP008 for 24 hours before being exposed to 100 μM glutamate for 24 hours. Cell viability was assessed by MTT assay. The statistical analysis was preformed using one-way ANOVA followed by Dunnett's test. Mean±SD, n=6. ** p<0.01, *** p<0.001 compared to 0 UM. ^{xxx} p<0.001 compared to control group.

FIG. 6. Effect of procaine on HMG-CoA reductase mRNA synthesis on PC12 cells. PC12 cells were pre-incubated with 1 or 10 μM procaine for 18 hours before being exposed to Aβ₁₋₄₂ 1 μM for 24 hours. The expression of the HMG-CoA mRNA was measured at the end of the 24 hours period using a real-time quantitative PCR. The statistical analysis was performed using one-way ANOVA followed by Dunnett's test. Mean±SD, n=3. * p<0.05,** p<0.01 compared to control unless differently specified.

### DETAILED DESCRIPTION OF THE INVENTION

Local anesthetics have been shown to exhibit neuroprotective properties in vivo, during cerebral ischemia in gerbils [Fujitani et al. (1994), Neurosci. Lett., 179:91-4; Chen et al. (1998) Brain Res., 4:16; Adachi et al. (1999) Brit. J. Anaesth; 83:472, and in vitro, during an hypoxic episode in hippocampal neurons [Lucas et al. (1989) J. Neurosci. Methods, 28:47; Liu et al. (1997) Anesthesiology, 87:1470; Raley-Susman et al., (2001) J. Neurophysiol. 86:2715-26.]. Concomitantly, procaine and lidocaine have been show to inhibit NMDA receptor activity [Nishizawa et al., (2002) Anesth. Analg., 94:325-30,], suppress the anoxia-induced increase of the intracellular calcium concentration in gerbil hippocampus [Liu et al., (1997) Anesthesiology, 87:1470] and prevent the ischemia-triggered increase of extracellular glutamate concentration in gerbil brain [Fujitani et al., 1994, cited above].

As used herein, the term “treatment of Alzheimer's disease” includes inhibiting the development of AD in a subject exhibiting at least one of the symptoms of the onset of AD, or who is likely to develop AD, as well as the ability to halt or slow the progression of AD, or to reduce or alleviate at least one of the symptoms of AD. The term “treatment” as used with respect to any neuropathology, such as multiple sclerosis, vascular dementia, age-related depression and mood swings and the like, is also intended to be defined in this manner.

The following definitions are used, unless otherwise described: halo is fluoro, chloro, bromo, or iodo. Alkyl, alkoxy, alkenyl, alkynyl, etc. denote both straight and branched groups; but reference to an individual radical such as Apropyl@ embraces only the straight chain radical, a branched chain isomer such as Aisopropyl@ being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing about 5 or 6 ring atoms consisting of carbon and one to four heteroatoms each selected from the group consisting of non-peroxide oxygen, sulfur, and N(R⁷) wherein R⁷ is absent or is as defined above; as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine anti-toxin activity using the standard tests described herein, or using other similar tests which are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₁₂)cycloalkyl can be monocyclic, bicyclic or tricyclic and includes cyclopropyl, cyclobutyl, cyclopentyl, cyclohexyl, bicyclo[2.2.2]octanyl, norbornyl, adamantyl as well as various terpene and terpenoid structures. (C₃-C₁₂)cycloalkyl(C₁-C₆)alkyl includes the foregoing cycloalkyl and can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl. Heterocycloalkyl and (heterocycloalkyl)alkyl include the foregoing cycloalkyl wherein the cycloalkyl ring system is monocyclic, bicyclic or tricyclic and optionally comprises 1-2 S, non-peroxide O or N(R⁷) as well as 2-12 ring carbon atoms; such as morpholinyl, piperidinyl, piperazinyl, indanyl, 1,3-dithian-2-yl, and the like; The cycloalkyl ring system optionally includes 1-3 double bonds or epoxy moieties and optionally is substituted with 1-3 OH, (C₁-C₆)alkanoyloxy, (CO), (C₁-C₆)alkyl or (C₂-C₆)alkynyl. (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be formyl, acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be alkyl substituted with 1 or 2 OH groups, such as hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 3,4-dihydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, indanyl, or naphthyl; and heteroaryl can be furyl, imidazolyl, triazolyl, triazinyl, oxazolyl, isoxazolyl, thiazolyl, isothiazolyl, pyrazolyl, pyrrolyl, pyrazinyl, tetrazolyl, pyridyl, (or its N-oxide), thienyl, pyrimidinyl (or its N-oxide), 1H-indolyl, isoquinolyl (or its N-oxide) or quinolyl (or its N-oxide).

Local or topical anesthetics, all of which are believed to be useful in the present invention, are an art-recognized class of drugs which temporarily interrupt mammalian nerve transmissions. They can generally be grouped into three chemical classifications structurally; the N-arylamides or carboxamides, such as lidocaine; the aminoalkylbenzoates, such as procaine, benoxinate and proparacaine, and the aminoalkylbenzamides, such as procainamide. Preferred N-arylamides comprise the N—(C₇-C₂₂)arylamides of amino-substituted (C₁-C₅)carboxylic acids, e.g., N-[(mono or di-(C₁-C₄)alkyl)phenyl]amides of aliphatic (C₁-C₅)carboxylic acids, which acids are preferably substituted with the moiety (R⁷)(R⁸)N—, wherein R⁷ is H or (C₁-C₅)alkyl and R⁸ is (C₁-C₅)alkyl. For example, a preferred carboxylic acid can have the general formula (R⁷)(R⁸)N(X)CO₂H where R⁷ and R⁸ are as defined above and X is a branched- or straight-chain (C₁-C₅)alkylene group such as 1,1-ethylene, 1,2-ethylene, methylene, 2,2-propylene, 1,3-propylene, and the like. Another preferred class of N-arylamides are the N-(mono- or di-(C₁-C₄) alkyl)phenyl]amides of 5- or 6-membered-heterocycloaliphatic carboxylic acids, which acids comprise one or two [(C₁-C₄)alkyl-substituted]N atoms, i.e., N-butylpiperidine-2-carboxylic acid.

Useful topical anesthetics of this class include lidocaine ((2-diethylamino)-N-(2,6-dimethylphenyl)-acetamide) (see Lofgren et al. (U.S. Pat. No. 2,441,498), May & Baker (British Patent No. 706409) and Macfarlane & Co. (British Patent No. 758,224)); bupivacaine (1-butyl-N-(2,6-dimethylphenyl)-2-piperidinecarboxyamide) (see Thuresson et al., (U.S. Pat. No. 2,955,111) and Sterling Drug (British Patent Nos. 1,166,802 and 1,180,712)); mepivacaine (2-piperidinecarboxyamide, N-(2,6-dimethylphenyl)-1-methyl), etidocaine (N-(2,6-dimethylphenyl)-2-(ethylpropylamino)butanamide; see, Astra (German Patent No. 2162744)); dibucaine (3-butoxy-N-[2-(diethylamino)ethyl]-4-quinolinecarboxyamide; Miescher (U.S. Pat. No. 1,825,623)); dyclonine (1-(4-butoxyphenyl)-3-(1-piperidinyl-1-propanone)); prilocalne (N-(2-methylphenyl)-2-(propylamino)propanamide); pyrrocaine (1-(pyrrolidin-1-yl)-N-(2,6-dimethylphenyl)acetamide, dimethyisoquin, diperodon, cocaine and its analogs (see, Carroll et al., J. Med. Chem., 34, 2719 (1991); Eur. J. Pharmacol., 184, 329 (1990); and the pharmaceutically acceptable salts thereof.

The aminoalkylbenzoates include esters between benzoic acids and alcohols of the general formula (R⁷)(R⁸)N(Alk)OH, wherein Alk is as defined above. R⁷ is H or (C₁-C₄)-alkyl, R⁸ is (C₁-C₄)alkyl or R⁷ and R⁸ taken together with N are a 5- or 6-membered heterocyclic ring, optionally substituted by (C₁-C₃)alkyl or comprising an additional ring O— or N(R⁷)-atom. The benzoic acid moiety can be the moiety (R⁹)(R¹⁰)ArCO₂H wherein Ar is an aromatic —C₆H₂₋₄ radical “phenylene” and each R⁹ and R¹⁰ is individually H, halo, preferably Cl; (R⁵)(H)N—, H₂N— or (C₁-C₅)alkoxy. Ar can also be (C₆-C₁₂) heteroaryl, optionally substituted with R⁹ and R¹⁰.

Useful topical anesthetics including chloroprocaine (4-amino-2-chlorobenzoic acid 2-(diethylamino)ethyl ester); procaine (4-aminobenzoic acid 2-(diethylamino)ethyl ester); tetracaine (4-(butylamino)benzoic acid 2-(dimethylaminoethyl ester; see Shupe (U.S. Pat. No. 3,272,700)); benoxinate (4-amino-3-butoxybenzoic acid 2-(diethylamino)ethyl ester (U.K. Patent No. 654,484)) proparacaine (3-amino-4-propoxybenzoic acid 2-(diethylamino)ethyl ester); isobucain (1-propanol, 2-methyl-2-[(2-methylpropyl)amino]benzoate; meprylcaine ([(2-methyl)(2-propylamino)propyl]benzoate; piperocaine ((2-methylpiperidin-1-ylpropyl(benzoate)); propoxycaine (2-(diethylamino)ethyl-([2′-methyl-4′-amino]benzoate)); butacaine (((3-dibutylamio)propyl)-(2′-aminobenzoate)); cyclomethylcaine (((3-2′-methylpiperidine-1-yl))propyl)-[4′-cyclohexyloxy-benzoate]); hexylcaine (([2-cyclohexylamino)(1-methyl)]ethyl)(benzoate) and proparacaine (((2-diethylamino)ethyl) [(4′-propyloxy-3′-amino)benzoate]).

Preferred salts include the amino addition salts of inorganic and organic acids, e.g., the hydrochloride, hydrobromide, sulfate, oxalate, fumarate, citrate, malate, propionate and phosphate salts. The hydrochloride and sulfate salts are preferred for use in the present invention.

These topical anesthetics and the salts thereof are discussed in detail in Remington's Pharmaceutical Sciences, A. Osol, ed., Mack Pub. Co., Easton, Pa. (16^{th} ed. 1980), and in The Merck Index (11^{th} ed. 1989).
A specific value for R¹ in formula I or II, above is H, (C₂-C₄)alkyl, (C₂-C₄)alkoxy, (C₃-C₆)cycloalkoxy, or (C₃-C₆)heterocycloalkyl.
A specific value for R² is H.
A specific value for R³ is H.
A specific value for R⁴ is H or N(R⁵)(R⁶), which is preferably is amino or (C₁-C₄)alkylamino.
A specific value for N(R⁷)(R⁸) is dimethylamino, diethylamino, dipropylamino, cyclohexylamino, or propylamino.
A specific value for (Alk) is —(CH₂)₁₋₃—.

A preferred group of compounds are compounds of formula II which are aminoalkyl benzoates.

Another preferred group of compounds are compounds of formula II which are N-aminoalkyl-benzamides, or (N-aryl)alkylbenzamides.

A preferred compound of the invention is lidocaine, procaine, tetracaine or procainamide, or an analog thereof.

Benzamide compounds of formula II can be prepared as shown in Scheme A, below.

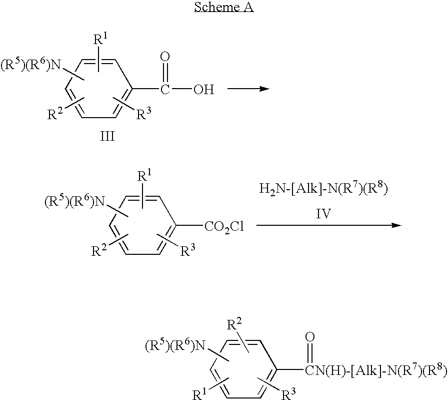

Benzoates can be prepared by replacing amine III with the corresponding alcohol and using it to esterify III. Groups R¹, R² and/or R³ on phenyl that are reactive with SOCl₂, or (C(O)Cl)₂ such as hydroxy-containing or thio-containing groups can be protected with removable protecting groups such as ethyoxyethyl, THP, (C₁-C₄)₃silyl and the like. Protected OH and hydroxylalkyl groups can be deprotected, and converted into halo, CN, alkoxycarbonyl, alkanoyloxy and alkanoyl by methods known to the art of organic synthesis. Protected amino groups can be deprotected and converted into N(R⁶)(R⁷) by methods known to the art. If necessary the C═O group can be protected and/or reduced during these conversions, then deprotected and reoxidized to C═O. See, for example, I. T. Harrison, Compendium of Organic Synthetic Reactions, Wiley-Interscience, N.Y. (1971); L. F. Fieser et al., Reagents for Organic Synthesis, John Wiley & Sons, Inc., N.Y. (1967), and U.S. Pat. No. 5,411,965.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium), alkaline earth metal (for example calcium or magnesium) or zinc salts can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes, or by inhalation or insufflation.

Thus, the present compounds may be systemically administered, e.g., orally, in combination with a pharmaceutically acceptable vehicle such as an inert diluent or an assimilable edible carrier. They may be enclosed in hard or soft shell gelatin capsules as powders, pellets or suspensions or may be compressed into tablets. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices, such as patches, infusion pumps or implantable depots.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection, infusion or inhalation can include sterile aqueous solutions or dispersions. Sterile powders can be prepared comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate, cellulose ethers, and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 mg to as much as 1-3 g, conveniently 10 to 1000 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline. For example, as much as about 0.5-3 g of a compound of formula I can be dissolved in about 125-500 ml of an intravenous solution comprising, e.g., 0.9% NaCl, and about 5-10% glucose. Such solutions can be infused over an extended period of up to several hours, optionally in conjunction with other anti-viral agents, antibiotics, etc. The active ingredient can also be orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound of the invention to act as an antiviral agent may be determined using pharmacological models which are well known to the art, or using tests described below.

The following illustrate representative pharmaceutical dosage forms, containing a compound of formula I, for therapeutic or prophylactic use in humans.

| (i) Tablet 1 | mg/tablet |
|---|---|
| Procainamide | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Tetracaine | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Tetracaine | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Lidocaine | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Procaine | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |

| (vi) Aerosol | mg/can |
|---|---|
| Lidocaine | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

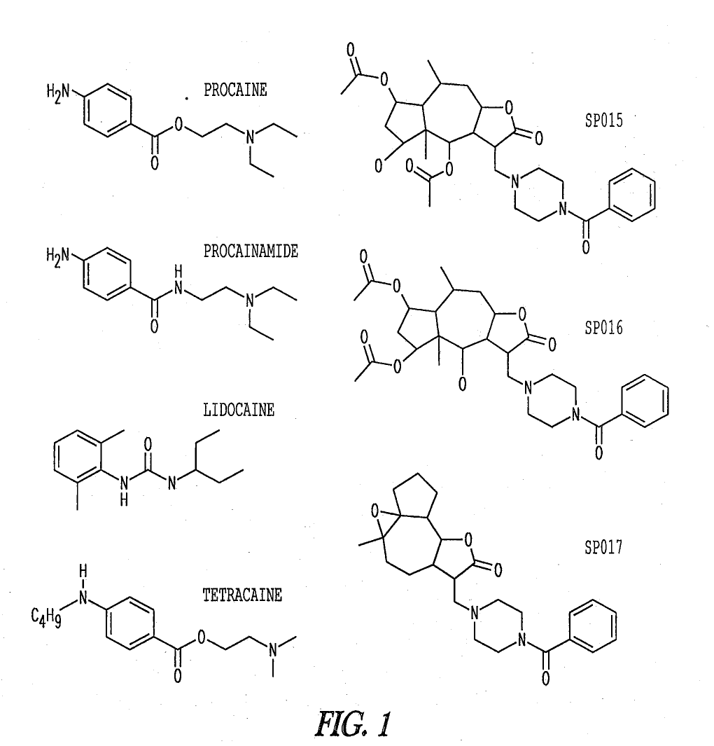

The invention will be further described by reference to the following detailed examples, wherein Aβ₁₋₄₂ peptide was purchased from American Peptide Co. (Sunnyvale, Calif.). Procaine, tetracaine, lidocaine, procainamide, the antioxidant tert-butyl-phenylnitrone (PBN), the N-methyl-D-aspartate (NMDA) receptor antagonist (+)-M801, and tetrodotoxine (TTX) were purchased from Sigma (St. Louis, Mo.). Structures of procaine, tetracaine, lidocaine, procainamide SP015, SP016 and SP017 are shown in FIG. 1. Cell culture supplies were purchased from GIBCO (Grand Island, N.Y.) and cell culture plasticware was from Corning (Corning, N.Y.) and Packard BioSciences Co. (Meriden, Conn.). RNA STAT-60 was from TEL-TEST, Inc. (Friendswood, Tex.). TaqMan® Reverse Transcription Reagents, random hexamers, and SYBR® Green PCR Master Mix were from Applied Biosystems (Foster City, Calif.).

### Methodology

### A. In Silico Screening for Procaine Derivatives

The Interbioscreen Database of naturally occurring entities was screened for compounds containing the procaine structure using the ISIS software (Information Systems, Inc., San Leandro, Calif.). Acetic acid 7-acetoxy-3-(4-benzoyl-piperazin-1-yl-methyl)-5-hydroxy-4a, 8-dimethyl-2-oxo-dodecahydro-azuleno[6,5-b]furan-4-yl ester (SP015), acetic acid 5-acetoxy-3-(4-benzoyl-piperazin-1-yl-methyl)-4-hydroxy-4a, 8-dimethyl-2-oxo-dodecahydro-azuleno[6,5-b]furan-7-yl ester (SP016) and 3-(4-benzoyl-piperazin-1-yl-methyl)-6,6a-epoxy-6,9-dimethyl-3a,4,5,6,6a,7,9a,9b-octahydro-3H-azuleno[4,5-b]furan-2-one (SP017) compounds identified were purchased from Interbioscreen (Moscow, Russia) (FIG. 1).

### B. Cell Culture and Treatments

PC12 cells (rat pheochromocytoma) (ATCC, Manassas, Va.) were cultured in RPMI 1640 without glutamine medium containing 10% of bovine serum and 5% of horse serum at 37° and 5% CO₂. These cells respond reversibly to NGF by induction of the neuronal phenotype. PC12 cells were incubated for 24 hours in 96-well plates (5.10⁴ cells per well) with increasing concentrations (1, 10 and 100 μM) of procaine, procainamide, lidocaine, tetracaine, SP015, SP016, SP017 or SP008. Aβ₁₋₄₂ was incubated overnight at 4° C. and then added to the cells at 0.1, 1 or 10 μM final concentrations for a 24 hours time period.

To study the role played by the NMDA receptor in the Aβ₁₋₄₂-induced neurotoxicity, increasing concentrations of (+)-MK801 were added to the cell media immediately before Aβ₁₋₄₂. Cell viability was assessed 4 hours later using the MTT assay. To assess the effect of procaine and SP008 on the glutamate-induced excitotoxicity, PC12 cells were pre-treated with procaine or SP008 at 0.3, 1, 3, 10 and 30 μM for 24 hours and then submitted to glutamate exposure for another 24 hour time period. Cell viability was subsequently assessed using the MTT assay. To assess the role of sodium channels in Aβ₁₋₄₂-induced neurotoxicity, PC12 cells were incubated for 4 hours with the sodium-channel blocker TTX at 3, 30 or 300 μM followed by addition of Aβ₁₋₄₂. Cell viability was assessed by MTT 24 hours later. The involvement of the oxidative stress in the toxicity of Aβ₁₋₄₂ was assessed by incubating the PC12 in the presence of 10, 100 or 500 μM PBN for 24 hours. Aβ₁₋₄₂ was then added to the incubation media. Cell viability was assessed by MTT 24 hours later.

### C. Cell Viability Determination

The cellular toxicity of Aβ was assessed using the 3-(4,5-dimethylthiazol-2-yl)-2,5-diphenyl tetrazolium bromide (MTT) assay (Trevigen, Gaithersburg, Md.) as previously described [Lecanu et al. (2004) Steroids, 69:1-16.]. Briefly, 10 μl of the MTT solution were added to the cells cultured in 100 μl of medium. After an incubation period of 4 hours in the same conditions as above, 100 μl of detergent were added and cells incubated overnight at 37° C. The blue coloration was quantified at 600 nm and 690 nm using the Victor spectrophotometer (EGG-Wallac, Gaithersburg, Md.). The effect of Aβ₁₋₄₂ was expressed as (DO₆₀₀-DO₆₉₀). To compare the protective effect of the compounds tested, the decrease of MTT signal observed with Aβ₁₋₄₂ was considered to be the 100% inhibition of the NADPH diaphorase activity and the effect of the compounds tested is shown as an increase or decrease of this percentage.

### D. ATP Measurement

ATP concentrations were measured using the ATPLite-M™ assay (Packard BioSciences Co.), as previously described [Lecanu et al., cited above]. In brief, cells were cultured on black 96-well ViewPlate™ and the ATP concentrations measured on a TopCount NXT™ counter (Packard BioSciences Co.) according to the manufacturer recommendations. The effect of Aβ₁₋₄₂ was expressed in arbitrary units. To compare the potential protective effect of the compounds tested on ATP recovery, the decrease of ATP concentration induced by Aβ₁₋₄₂ was considered to be 100% reduction and the effects of the compounds tested are shown as changes of this percentage.

### E. Free Radical Production

Oxidative stress was assessed by measuring the free radical production using the fluorescent probe di-hydroxy di-chlorofluorescein diacetate (2,7-DCF) (Molecular Probes, Eugene, Oreg.), as previously described [Lecanu et al., cited mix (5 μM each) with 2 μl cDNA. The cycling conditions were: 15 seconds at 95° C. and 1 minute at 60° C. for 40 cycles following an initial step of 2 minutes at 50° C. and 10 minutes at 95° C. AmpliTaq Gold polymerase was activated at 95° C. for 10 minutes. The 18S RNA was amplified at the same time and used as an internal control. To exclude the contamination of unspecific PCR products such as primer dimmers, a melting curve analysis was applied to all final PCR products after the cycling protocol. Also, PCR reactions without the RT reaction were performed for each sample in order to exclude genomic DNA contamination. The PCR products were collected and run on a 3% (w/v) agarose/TAE gel to confirm the product size. The threshold cycle (Ct) values for 18S RNA and samples were calculated using the PE/AB computer software. Ct was determined at the most exponential phase of the reaction. Relative transcript levels were calculated as x=2^{ΔΔCt}, in which ΔΔCt=ΔE−ΔC, and ΔE=Ctₑₓₚₑᵣᵢₘₑₙₜ−Ct_{18S}, ΔC−Ct_{control}−Ct_{18S}. H.

### H. Statistical Analysis

Data are expressed as mean±SD. Data obtained were assessed between experimental groups by a one-way ANOVA and Dunnett's test was used for comparison. A difference was considered significant when p<0.05.

### Example 1

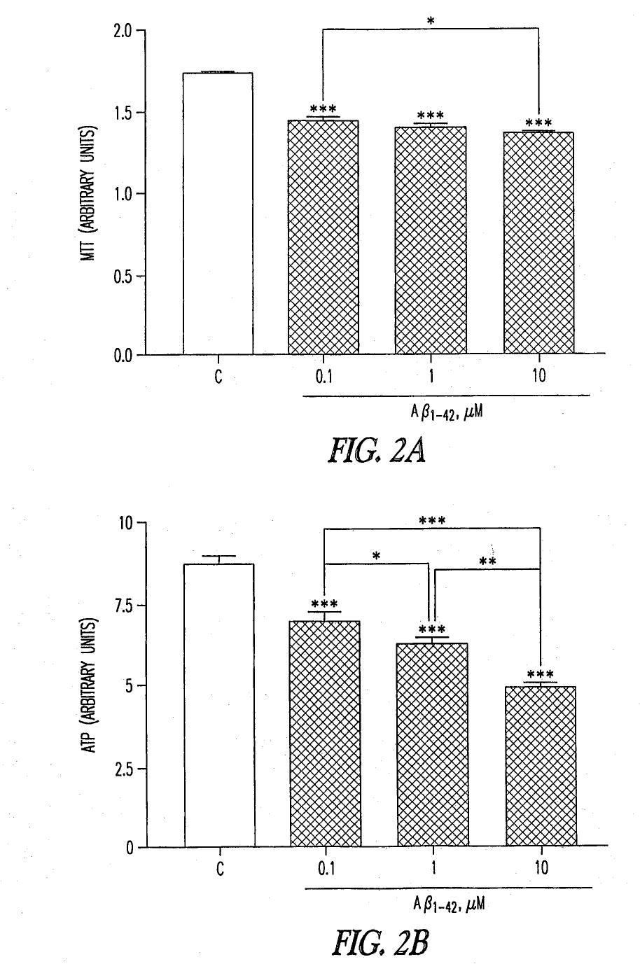

### Aβ₁₋₄₂ Neurotoxicity Assessed by MTT Assay, ATP Measurement and Free Radical Production in PC12 Cells (FIG. 2)

Aβ₁₋₄₂ induces a dose-dependent decrease of PC12 cell viability (p<0.001) (FIG. 2A) and of the intra-cellular ATP concentrations (p<0.001) (FIG. 2B). A dose-dependent relationship is also observed on the free radical production as Aβ₁₋₄₂ at 1 and 10 μM concentrations induced a significant increase of the oxidative stress (p<0.01 and p<0.001 respectively) (FIG. 2C).

### Example 2

### Effect of the Procaine and the Procaine Derivatives on the Cell Viability Assessed by MTT

As shown on Table 1, procaine displays an important protective effect against 0.1 and 1 μM Aβ₁₋₄₂ induced toxicity assessed using the MTT assay.

TABLE 1
Assessment of the neuroprotective effect of the SP compounds against Aβ₁₋₄₂ cytotoxicity on PC12 cells

| | | Procaine | | | Lidocaine | | |
|---|---|---|---|---|---|---|---|
| Aβ 1-42 | Control | 1 | 10 | 100 | 1 | 10 | 100 |
| 0.1 | 100.0 ± 8.8 | 70.0 ± 13.7** | 70.3 + 19.0** | 91.5 ± 2.1** | 80.1 ± 11.5* | 83.3 ± 15.3 | 81.5 ± 10.0 |
| 1 | 100.0 ± 6.6 | 70.1 ± 22.4** | 62.5 + 12.2** | 92.5 ± 15.8 | 68.9 ± 15.4** | 73.1 ± 14.9* | 76.2 ± 18.8 |
| 10 | 100.0 ± 5.3 | 114.5 ± 9.9 | 100.6 ± 7.8 | 86.2 ± 5.1* | 71.2 ± 16.6** | 72.5 ± 15.4** | 76.4 ± 22.2 |

| | | Tetracaine | | | Procainamide | | |
|---|---|---|---|---|---|---|---|
| Aβ 1-42 | Control | 1 | 10 | 100 | 1 | 10 | 100 |
| 0.1 | 100.0 ± 8.8 | 89.0 ± 10.2 | 91.1 ± 6.6 | 60.1 ± 8.2** | 73.7 ± 11.6** | 68.0 ± 11.6** | 86.1 ± 13.4 |
| 1 | 100.0 ± 6.6 | 87.1 ± 12.2 | 86.0 ± 6.6 | 43.7 ± 7.6*** | 72.1 ± 10.9* | 49.3 ± 8.0*** | 69.2 ± 15.7** |
| 10 | 100.0 ± 5.3 | 77.4 ± 11.6* | 84.4 ± 9.9* | 39.6 ± 16.6*** | 79.9 ± 6.5** | 51.4 ± 16.7*** | 73.6 ± 12.4** |

| | | SP015 | | | SP016 | | |
|---|---|---|---|---|---|---|---|
| Aβ 1-42 | Control | 1 | 10 | 100 | 1 | 10 | 100 |
| 0.1 | 100.0 ± 8.8 | 86.6 ± 19.6 | 95.9 + 9.0 | 115 ± 14.7 | 88.1 ± 25.5 | 89.6 ± 31.6 | 124 ± 26.8 |
| 1 | 100.0 ± 6.6 | 78.8 ± 24.7 | 82.8 + 21.9 | 116 ± 22.3 | 87.2 ± 32.5 | 86.1 ± 25.9 | 126 ± 29.6 |
| 10 | 100.0 ± 5.3 | 78.1 ± 15.0* | 78.5 ± 16.6* | 120 ± 25.1 | 88.2 ± 23.6 | 78.1 ± 43.9 | 118 ± 34.3 |

| | | SP017 | | |
|---|---|---|---|---|
| Aβ 1-42 | Control | 1 | 10 | 100 |
| 0.1 | 100.0 ± 8.8 | 94.1 ± 8.5 | 70.1 ± 21.9* | 208 ± 10.0 |
| 1 | 100.0 ± 6.6 | 65.7 ± 10.7*** | 71.9 ± 14.6** | 231 ± 11.1 |
| 10 | 100.0 ± 5.3 | 54.6 ± 20.6** | 69.4 ± 21.7* | 226 ± 11.1 |

Data presented as s.e.m. ± SD (n = 6).
*p < 0.05,
**p < 0.01,
***p < 0.001 compared to control.
Statistical analysis performed by ANOVA followed by a Dunnett's test.

Treatment with 1 and 10 μM procaine resulted in a reduction of the NADPH diaphorase inhibition induced by Aβ₁₋₄₂ of at least 30% (p<0.01); at higher concentrations procaine was less effective. Lidocaine reduced significantly the NADPH diaphorase inhibition when used at 1 μM even against the highest concentration of Aβ₁₋₄₂ (71.2±16.6% compared to the control 100.0±5.3%, n=6, p<0.01). Lidocaine at 10 μM provided a protection equivalent to that observed with 1 μM except against the lowest dose of Aβ₁₋₄₂; again the concentration of 100 μM lidocaine was less efficacious than the 1 and 10 μM concentrations and without effect against 10 μM Aβ₁₋₄₂. The three concentrations of tetracaine protected against 10 μM Aβ₁₋₄₂ with the strongest effect observed by 100 μM tetracaine (39.6±16.6% compared to control 100.0±5.3%, P<0.001, n=6). Only this tetracaine concentration was able to reduced the NADPH diaphorase inhibition induced by Aβ₁₋₄₂ 0.1 and 1 μM with respectively 60.1±8.2% versus 100.0±8.8% for the control (p<0.01, n=6) and 43.7±7.6% versus 100.0±6.6% for the control (p<0.001, n=6). The three concentrations of procainamide used dramatically reduced the NADPH diaphorase inhibition induced by Aβ₁₋₄₂ except the 100 μM concentration against 0.1 μM Aβ₁₋₄₂. The highest level of neuroprotection was observed with 10 μM procainamide and was equivalent to the result obtained with 100 μM tetracaine with respectively 68.0±11.6% versus 100.0±8.8% for the control (p<0.01, n=6), 49.3±8.0% versus 100.0±6.6% for the control (p<0.001, n=6) and 51.4±16.7% versus 100.0±5.3% for the control (p<0.001, n=6).

The identified naturally occurring procaine derivatives also displayed neuroprotective properties against Aβ₁₋₄₂ neurotoxicity in PC12 cells but at concentrations different to those reported above for procaine. SP015 protected only at 1 and 10 μM concentrations against the highest concentration of Aβ₁₋₄₂, whereas SP016 had no protective activity. SP017 at 1 μM reduced the diaphorase inhibition induced by Aβ₁₋₄₂ but the best effect was observed with SP017 10 μM which was able to protect against the three concentrations of Aβ₁₋₄₂ tested (70.1±21.9% versus 100.0±8.8% for the control, p<0.05, n=6; 71.9±14.6% versus 100.0±6.6% for the control, p<0.01, n=6, and 69.4±21.7% versus 100.0±5.3% for the control, p<0.05, n=6. SP017 at 100 μM potentiated the toxic effect of Aβ₁₋₄₂ suggesting a probable toxicity.

### Example 3

### Effect of the Procaine and Procaine Derivatives on Aβ₁₋₄₂-Induced ATP Decrease

As shown in Table 2, procaine protected against the 0.1 μM Aβ₁₋₄₂-induced depletion of ATP concentrations in a dose-dependent manner, whereas its protective effect was less consistent against 1 μM Aβ₁₋₄₂ and did not occur against 10 μM Aβ₁₋₄₂.

TABLE 2
SP compounds reverse the ATP stock depletion induced by Aβ₁₋₄₂ on PC12 cells

| | | Procaine | | | Lidocaine | | |
|---|---|---|---|---|---|---|---|
| Aβ 1-42 | Control | 1 | 10 | 100 | 1 | 10 | 100 |
| 0.1 | 100.0 ± 22.9 | 64.0 ± 24.4* | 56.0 ± 16.4** | 42.8 ± 16.9** | 38.7 ± 26.1** | 47.2 ± 28.7** | 65.3 ± 45.2 |
| 1 | 100.0 ± 15.9 | 67.8 ± 13.8** | 97.0 ± 45.5 | 69.6 + 12.1** | 62.0 ± 8.5** | 69.0 ± 13.0** | 81.3 ± 8.7 |
| 10 | 100.0 ± 23.4 | 86.8 ± 5.5 | 89.8 ± 5.1 | 83.6 ± 20.6 | 53.5 ± 20.3** | 65.0 ± 7.6** | 70.1 ± 4.8* |

| | | Tetracaine | | | Procainamide | | |
|---|---|---|---|---|---|---|---|
| Aβ 1-42 | Control | 1 | 10 | 100 | 1 | 10 | 100 |
| 0.1 | 100.0 ± 22.9 | 54.8 ± 4.0** | 59.5 ± 9.6** | 59.3 ± 10.5* | 46.8 ± 25.9** | 60.4 ± 5.8** | 40.9 ± 14.5** |
| 1 | 100.0 ± 15.9 | 32.2 ± 5.5** | 58.0 ± 18.9** | 45.4 ± 2.2** | 51.4 ± 17.3** | 61.7 ± 10.8** | 40.8 ± 6.5** |
| 10 | 100.0 ± 23.4 | 45.5 ± 6.1** | 41.9 ± 4.2** | 45.8 ± 6.5** | 56.5 ± 11.6** | 56.2 ± 6.1** | 52.2 ± 10.2** |

| | | SP015 | | | SP016 | | |
|---|---|---|---|---|---|---|---|
| Aβ 1-42 | Control | 1 | 10 | 100 | 1 | 10 | 100 |
| 0.1 | 100.0 ± 13.4 | 46.3 ± 10.1** | 99.2 ± 13.1 | 91.5 ± 1.5 | 49.5 ± 10.2** | 101 ± 7.8 | 86.3 ± 15.7 |
| 1 | 100.0 ± 18.9 | 55.6 ± 5.3** | 124 ± 24.7 | 85.6 ± 12.3 | 81.5 ± 4.2 | 105 ± 10.1 | 100 ± 17.8 |
| 10 | 100.0 ± 7.3 | 32.8 ± 9.4** | 104 ± 22.5 | 73.8 ± 6.8* | 96.8 ± 30.6 | 110 ± 10.1 | 121 ± 29.0 |

| | | SP017 | | |
|---|---|---|---|---|
| Aβ 1-42 | Control | 1 | 10 | 100 |
| 0.1 | 100.0 ± 13.4 | 28.1 ± 7.2** | 54.9 ± 15.6** | 115 ± 21.4 |
| 1 | 100.0 ± 18.9 | 42.6 ± 5.8** | 44.9 ± 8.1** | 108 ± 20.8 |
| 10 | 100.0 ± 7.3 | 73.0 ± 12.1* | 68.9 ± 7.3** | 122 ± 6.7 |

Data presented as s.e.m. ± SD (n = 6).
*p < 0.05,
**p < 0.01,
*** p < 0.001 compared to control.
Statistical analysis performed by ANOVA followed by a Dunnett's test.

Lidocaine tested at 1 and 10 μM restored ATP concentrations in PC12 cells exposed to 0.1 and 1 μM Aβ₁₋₄₂ (p<0.01, n=6) with the most important effect observed against 0.1 μM Aβ₁₋₄₂. Lidocaine tested at the concentration of 100 μM exerted a protective effect against all concentrations of Aβ₁₋₄₂, although this effect was statistically significant only against 10 μM Aβ₁₋₄₂ with 100.0±23.4% (p<0.05, n=6). The three concentrations of tetracaine and procainamide tested significantly prevented the Aβ₁₋₄₂-induced decrease of intracellular ATP levels.

Among the natural derivatives of procaine, SP015 at 1 μM and SP017 at 1 and 10 μM concentrations were able to reverse the effect of Aβ₁₋₄₂ on ATP.

### Example 4

### Effect of the NMDA Antagonist (+)-MK801 on Aβ₁₋₄₂-Induced Neurotoxicity

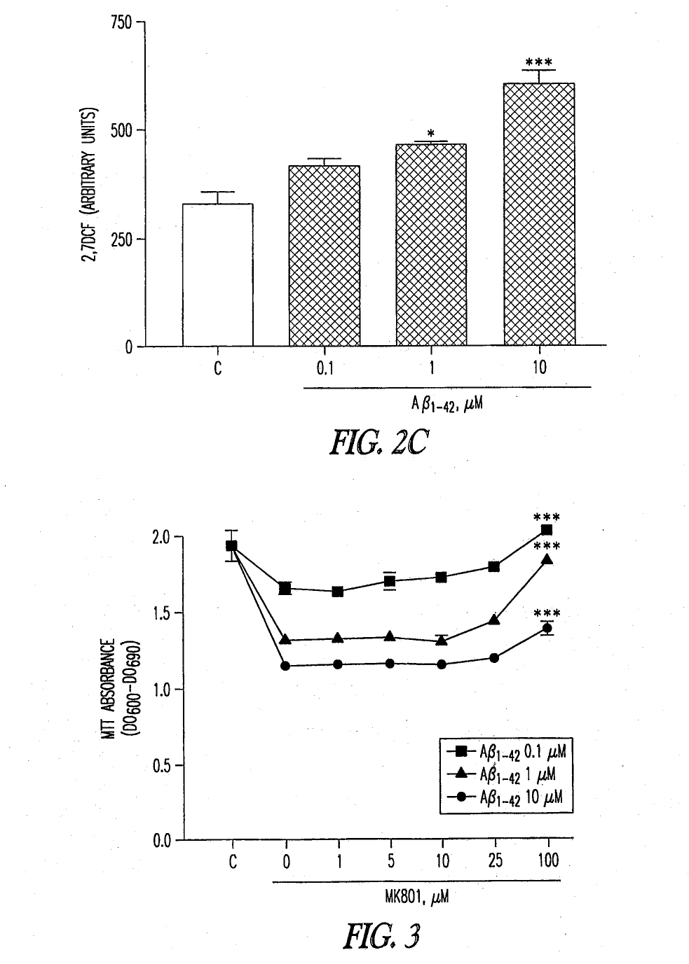

Procaine and others local anesthetics have been shown to inhibit the NMDA receptor and an over-activation of the NMDA receptor has been demonstrated to contribute to Aβ₁₋₄₂ neurotoxicity. Therefore, in order to assess if a neuroprotective effect of procaine could be due to the blockade of the NMDA neurotransmission, it was determined if a NMDA hyperactivity occurs in this experiment. This was studied by using (+)-MK801, a non-competitive inhibitor of NMDA receptor, on Aβ₁₋₄₂ neurotoxicity. (+)-MK801 lessens in a dose-dependent manner PC12 cell viability decrease induced by Aβ₁₋₄₂ (FIG. 3). (+)-MK801 used at 25 μM concentrations protected PC12 cells against 0.1 and 1 μM Aβ₁₋₄₂-induced toxicity (p<0.05). (+)-MK801 used at 100 μM concentrations provided the most significant neuroprotective effect against all concentrations of Aβ₁₋₄₂ tested (p<0.001).

### Example 5

### Displacement Study of the [³H](+)Pentazocine by Procaine on Sigma-1 Receptor

Because the sigma-1 receptor regulates or preserves important physiological functions or processes which are altered in AD, like calcium homeostasis, memory, mood and mitochondria functions, it is of interest to test the ability of procaine to bind this receptor. In order to do it, the displacement of the specific sigma-1 ligand pentazocine by procaine was measured. Procaine displaced the [³H](+)pentazocine from its binding site on the sigma-1 receptor expressed in Jurkat cells with an IC50 of 4.3 μM. The Hill coefficient (nH=1.0) revealed a single binding site for procaine on the sigma-1 receptor.

### Example 6

### Effect of Procaine and SP008 on Glutamate-Induced Excitotoxicity on PC12 Cells

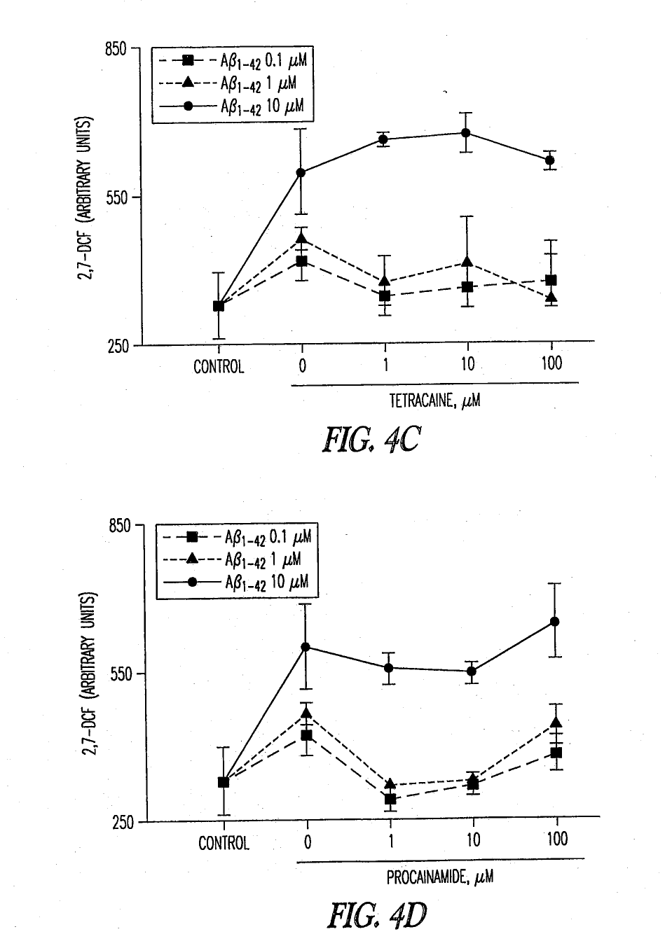

Glutamate 100 μM dramatically reduced PC12 cell viability (p<0.001, n=6; FIG. 5). Procaine prevented the glutamate-induced neurotoxicity in a biphasic manner. Two maximum effects were observed at 0.3 and 10 μM (p<0.001 compared to control, n=6). The SP008 effect was also biphasic reaching a protective peak at 3 μM (p<0.001 compared to control, n=6) followed by a decline in its neuroprotective property in the presence of at higher concentrations of glutamate. The neuroprotective effect of SP008 was more important than that of procaine effect at the same concentration (p<0.001, n=6).

### Example 7

### Effect of the Procaine and Procaine Derivatives on Aβ₁₋₄₂-Induced Free Radical Production

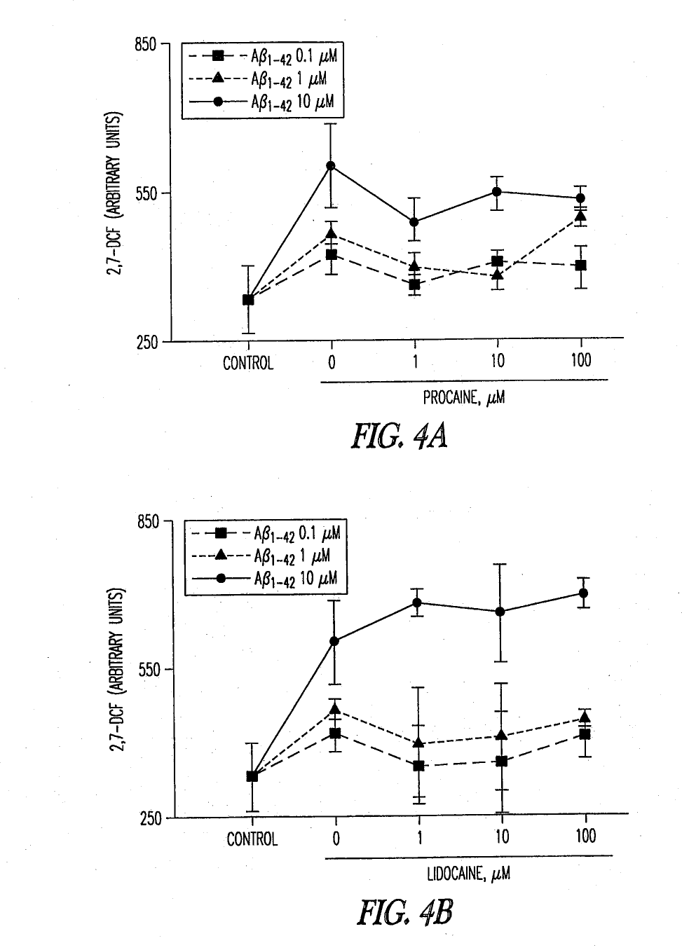

As shown in FIG. 2C Aβ₁₋₄₂-induced in a dose-dependent manner the production of free radicals in PC12 cells. Procaine (FIG. 4A), procainamide (FIG. 4B), lidocaine (FIG. 4C) and tetracaine (FIG. 4D) exhibited a trend to reduce the Aβ₁₋₄₂-induced free radical production. This effect was statistically significant in the presence of 10 μM procaine incubated with 1 μM Aβ₁₋₄₂ (p<0.05, n=6), 1 μM procaine when incubated with 0.1 μM Aβ₁₋₄₂ (p<0.05, n=6), 100 μM tetracaine when incubated with 1 μM Aβ₁₋₄₂ (p<0.05, n=6) and 1 and 10 μM procainamide when incubated with 0.1 and 1 μM Aβ₁₋₄₂ (p<0.01, n=6).

SP015, SP016 and SP017 compounds did not affect the Aβ₁₋₄₂-induced oxidative stress. On the contrary, these compounds amplified the Aβ₁₋₄₂-induced free radicals production.

### Example 8

### Effect of Procaine on HMG-CoA Reductase mRNA Synthesis on PC12 Cells

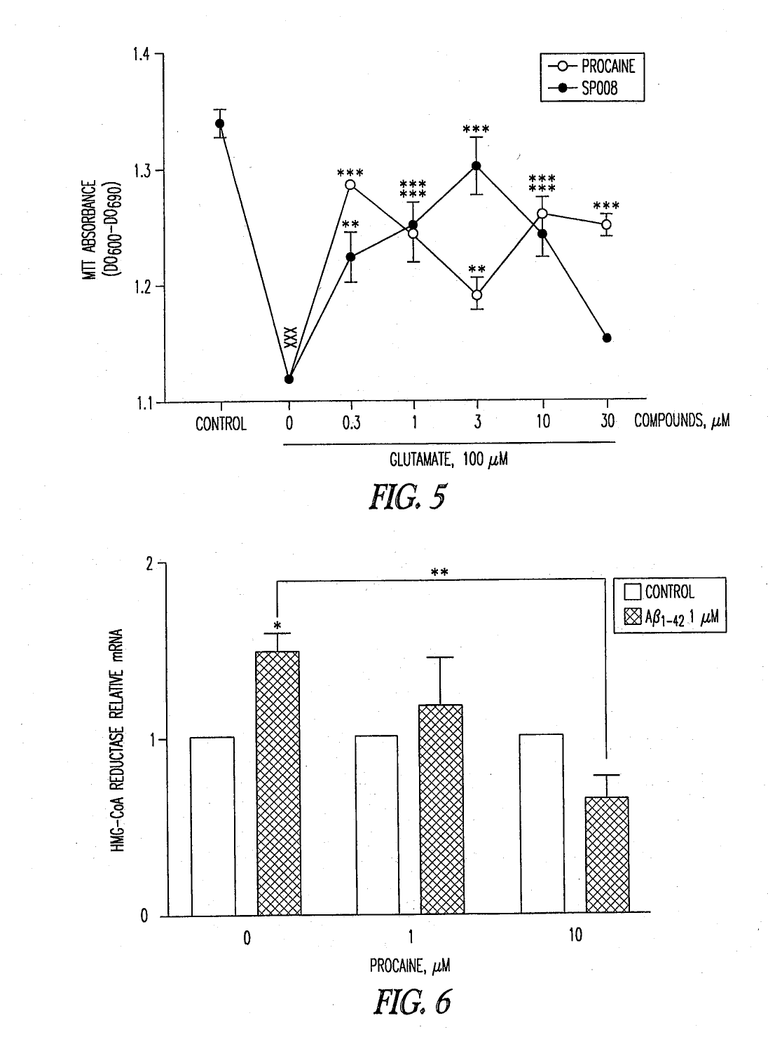

Aβ₁₋₄₂ (1 μM) induced a significant increase of HMG-CoA mRNA synthesis compared to the control PC12 cells (1.48±0.17 times the control level, p<0.05; FIG. 6). Procaine decreased in a dose-dependent manner the level of mRNA induced by Aβ₁₋₄₂ but did not affect the basal level of HMG-CoA reductase mRNA measured in control PC12.

### Discussion

During the past decades, improving the cholinergic network dysfunction associated with AD has been the main focus of the scientific community. This led to the creation of the therapeutic class of the acetylcholinesterase inhibitors (AchEI) with the tacrine as the class leader. Despite promising clinical data, the beneficial effects of tacrine were modest and the new generation of AchEI, represented by galantamine and donezepil, did not improve the delay of symptom onset compared to tacrine. This short 1-2 years delay, although priceless for the patients and their relatives, is probably due to the progressive degeneration of the cholinergic neurons and is a limitation of the use of AchEI. Even though the improvement of the cholinergic transmission of the patients suffering from AD is relevant and necessary, it is certainly not sufficient to stop or reverse the progression of the disease. Since, no major advance has been made in AD drug development, even though memantine, an antagonist of the glutamatergic NMDA-subtype receptor was recently approved to be released in the US market. The present invention provides a new class of compounds derived from the homologous domain of a series of natural compounds which were obtained by screening a database using procaine as a starting point. These molecules can protect rat pheochromocytoma PC12 cells against Aβ₁₋₄₂ neurotoxicity.

The adrenal hormone cortisol was described to worsen the AD evolution by enhancing the neuronal death, altering the mood and inducing depression and Xu et al recently reported that a procaine-based pharmaceutical preparation reduced the stress-induced hypercorticosteronism in rat [J. Pharmacol. Exp. Ther., 307:1148 (2003)], presenting therefore procaine as an interesting approach to treat AD. However, the quick degradation of procaine into para-aminobenzoic acid and diethylaminoethanol renders it difficult to use therapeutically for AD. SP015, SP016 and SP017 were obtained by screening natural compounds database using procaine as a sub-structure (FIG. 1) and they originate from plants of the Asteraceae family, Inula britanica and Artemisia glabella. Strikingly, plants from Artemisia genus have been used traditionally as restoratives of lost or declining mental functions [Wake et al., (2000) J. Ethnopharmacol. 69:105-14].

Procaine was able to restore partially the decrease of ATP production induced by Aβ₁₋₄₂ suggesting an activity on the mitochondrial respiratory chain. Among the screened natural compounds, SP017 showed the highest protective effect on the mitochondrial function, as evidenced by the changes seen in mitochondrial diaphorase activity, with efficacy range of 30-70% of inhibition of Aβ₁₋₄₂ toxicity. Interestingly, despite the important chemical similarity between SP015 and SP016, SP016 displayed a significant effect only against low Aβ₁₋₄₂ concentrations (0.1 μM) when administered at 1 μM whereas 1 μM SP015 offered an important protection even against the highest Aβ₁₋₄₂ concentration examined. Surprisingly, the effect of these different compounds on PC12 viability after Aβ₁₋₄₂ exposure did not completely match the effect observed on the restoration of ATP content. In particular, SP015 displayed a neuroprotective effect at 1 and 10 μM only against 10 μM Aβ₁₋₄₂ while no effect was observed with SP016. This apparent discrepancy suggests that the preservation of the intracellular ATP stock is not the only mechanism by which the procaine and procaine derivatives exert their neuroprotective properties.

The glutamatergic network is also targeted by the β-amyloid peptides since Aβ₁₋₄₀ [Wu et al., Neuroreport, 6, 2409 (1995)] and Aβ₂₅₋₃₅ [Mogensen et al., Neuroreport, 9, 1553 (1998)] have been described to selectively augment NMDA-receptor-mediated, but not AMPA, synaptic transmission in rat hippocampus. However, different results indicated that the non-NMDA receptor-evoked calcium inward current contributed to the neurotoxicity displayed by Aβ₁₋₄₂ on differentiated human NT2-N neurons [Blanchard et al., Brain Res., 21, 776(1-2):40 (1997)]. Thus, even though no data are available regarding an inhibitory effect of procaine on the AMPA/kainate receptors, the possibility that such a mechanism participated in the observed neuroprotection remains to be established.

Interestingly, the NMDA receptor antagonist MK-801 protected cholinergic nucleus basalis neurons and striatal neurons from amyloid peptide neurotoxicity in vivo [Parks et al., J. Neurochem., 76, 1050 (2001); Harkany et al., Eur. J. Neurosci., 12, 2735 (1999)] and in vitro on neuroblastoma cells, whereas AP-5, which binds specifically the glutamate site, did not [Le et al., Brain Res., 686, 49 (1995)]. These results led these authors to conclude that amyloid peptides might act more by stabilizing the opening state of the NMDA-associated calcium channel after inserting into the plasma membrane rather than by directly binding the glutamate site. Strikingly, the MK-801 reduced in a dose-dependent manner the neurotoxicity induced by Aβ₁₋₄₂ suggesting, therefore, the involvement of an over-stimulation of the NMDA receptors in the neurotoxicity discussed herein. Moreover, procaine reduced the glutamate-induced excitotoxicity on the PC12 cells, indicating that the inhibition of the NMDA-induced calcium inward current might account for the protective effect provided by the compounds of the invention. This data is reinforced by recent findings reporting that local anesthetic agents inhibit NMDA receptor channel in mouse CA1 pyramidal neurons [Nishizawa et al., Anesth. Analg., 94 325 (2002)] and in Xenopus oocytes [Sugimoto et al., Brit. J. Pharmacol., 138, 876 (2003)].

The mechanism by which the local anesthetics inhibit the NMDA receptor depends on their respective pKa. With a pKa of 8.9, procaine is the more ionized at physiological state and therefore, is probably more prone to bind a site located inside the calcium channel and to act in a voltage-dependent fashion. On the other hand, lidocaine has a pKa of 7.9, suggesting that this molecule exists essentially as a non-ionized lipophilic form at physiological pH and acts by inserting the plasma membrane and by allosterically modifying the NMDA receptor. With an intermediate pKa of 8.5, tetracaine is expected to inhibit the NMDA receptor by both mechanisms, which might therefore explain the highest efficacy of this compound in protecting PC12 cells against Aβ₁₋₄₂ neurotoxicity.

Such a mechanism of action might have accounted for the neuroprotective effect observed with the natural compounds SP015 and SP017 since they have been selected from databases using procaine as a substructure. Interestingly, an over-activation of the rat hippocampus NMDA receptors by Aβ₁₋₄₂ has been described to affect the long-term depression and, in turn, the long-term potentiation [Kim et al., J. Neurosci., 21, 1327 (2001)], the two main forms of synaptic plasticity in the brain. This deleterious pathway has been proposed to contribute to the memory processes hampered in AD.

Procaine further exhibits the ability to bind the sigma-1 (σ1) receptor with an IC₅₀ of 4.3 μM and a Hill coefficient of 1.0, indicating the presence of an unique binding site. Several σ1-receptor agonists have been described to reverse in a dose-dependent manner the scopolamine-induced amnesia in rats. Interestingly, one of them, the SA4503, enhanced the Ach release in the hippocampus of rat brain slices [Horan et al., Synapse, 46, 1 (2002)] and in vivo [Kobayashi et al., J. Pharmacol. Exp. Ther., 279, 106 (1996)], suggesting that the anti-amnesic effect could be due in part to the activation of the cholinergic pathway. In addition, the effect of the binding on the σ1-receptor on the Ach release seems to be much more pronounced in the hippocampus compared to tacrine [Kobayashi et al., J. Pharmacol. Exp. Ther., 279, 106 (1996)]. In addition, Igmesine, a σ1-receptor agonist, was recently demonstrated to exert an antidepressant activity in mice intracerebroventrically injected with the amyloid fragment Aβ25-35 [Urani et al., Behav. Brain Res., 134, 239 (2002)], probably via a modification of the monoaminergic system [Akunne et al., Neuropharmacol., 41, 138 (2001)]. In addition, recent data reported that σ1-receptor ligands protect neuronal cells against transient cerebral ischemia in rat [Goyagi et al., Anesth. Analg., 96, 532 (2003)], prevented the hypoxia-induced ATP depletion in astrocytes [Klouz et al., FEBS Lett., 553 157 (2003)] and facilitated neurite sprouting induced by nerve growth factor in PC12 cells [Takebayashi et al., J. Pharm. Exp. Ther., 303, 1227 (2002)]. Procaine bound selectively the σ1-receptor compared to the σ2-receptor (IC50>10 μM) and therefore it might be devoid of the pro-apoptotic properties and cytotoxic effect described for the σ2-receptor agonists.

Procaine was recently demonstrated to downregulate the stress-induced cortisol increase in vivo in rats and in vitro in dbcAMP-stimulated Leydig cells [Xu et al., J. Pharmacol. Exp. Ther., 307, 1148 (2003)]. The data reported indicated that the decrease of the cortisol production by the adrenal cortical cells was due to a decrease in the expression of cholesterol synthesis rate limiting enzyme HMG-CoA reductase mRNA and correlates with the restoration of cell viability. The effect of procaine on HMG-CoA mRNA levels in PC12 cells “stressed” by Aβ₁₋₄₂ exposure reported herein is equivalent to that previously reported by Xu et al. for adrenal cells “stressed” by cAMP. Interestingly, the ability of Aβ₁₋₄₂ to modulate the HMG-CoA activity through an increase of the expression of its mRNA is complementary to recent findings on the physiological function of the beta-amyloid peptide in the control of neuronal cholesterol levels and transport [Yao et al., Brain Res., 847, 203 (2002); Wood and Igbavboa, Pharmopsychiatry, 36(S2), 3144-148 (2003)].

However, it is very unlikely that any reduction of corticosteroid synthesis accounts for the protective effect of procaine against Aβ₁₋₄₂, as PC12 pheochromocytoma cells do not produce steroids. It is more likely that the dose-dependent reduction of HMG-CoA mRNA expression by procaine results, first, in a decrease of the cholesterol production with, as a direct consequence, a modification of the membrane fluidity and an alteration of Aβ₁₋₄₂ trafficking through the cell membrane. These modifications might therefore render the cell less sensitive to Aβ₁₋₄₂-induced neurotoxicity. In addition, the reduction of cholesterol synthesis has been shown to reduce APP cleavage and beta-amyloid peptide production by reducing γ-secretase activity.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for treatment of a mammal threatened or afflicted by a neuropathological condition by administering to said mammal an effective neuroprotective amount of a compound of formula I or formula II: 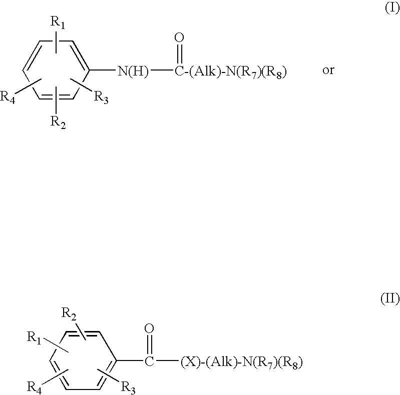
wherein:
a) R¹, R² and R³ are individually H, OH, halo, CN, (C₁-C₆)alkyl, (C₁-C₆)alkoxy, (C₃-C₆)cycloalkyl, (C₃-C₆)cycloalkoxy, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio, thio(C₁-C₆)alkyl-, (C₁-C₆)alkanoyloxy, N(R⁵)(R⁶) or R¹ and R² together are methylenedioxy;
b) R⁴ is hydrogen, (C₁-C₃)alkyl, N(R⁵)(R⁶), (C₁-C₆)alkoxy;
c) R⁵, R⁶, R⁷ and R⁸ are individually, H, (C₁-C₆)alkyl, (C₃-C₆)cycloalkyl, (C₃-C₆)cycloalkyl((C₁-C₆)alkyl), (C₂-C₆)alkenyl, wherein cycloalkyl optionally comprises 1-2, S, nonperoxide O or N(R⁵); aryl, aryl(C₁-C₆)alkyl, aryl(C₂-C₆)alkenyl, heteroaryl, heteroaryl(C₁-C₆)alkyl, or R⁵ and R⁶ or R⁷ and R⁸ together with the N to which they are attached form a 5- or 6-membered heterocyclic or heteroaryl ring, optionally substituted with R¹ and optionally comprising 1-2, S, non-peroxide O or N(R⁵);
d) (Alk) is (C₁-C₆)alkyl, (C₂-C₆)alkenyl, (C₃-C₆)cycloalkyl, (C₃-C₆)cycloalkyl(C₂-C₆)alkyl or [(C₂-C₆)alkyl(C₃-C₆)cycloalkyl[(C₃-C₆)alkyl] optionally substituted by 1-2 S, non-peroxide O or N(R⁵);
e) X is O or NH;
or a pharmaceutically acceptable salt thereof, with the proviso that two of R¹, R², R³ and R⁴ in formula (I) are not (C₁-C₃)alkyl.

2. The method of claim 1 wherein (Alk) is (C₁-C₄)alkyl, such as —(CH₂)—, —CH₂)₂—, —(CH₂)₃— or —(CH₂)₄—.

3. The method of claim 1 wherein 1 or 2 of R¹, R², R³ or R⁴ is N(R⁵)(R⁶).

4. The method of claim 1 wherein both of R⁵ and R⁶ is H.

5. The method of claim 1 wherein one or both of R⁷ and R⁸ are (C₁-C₆)alkyl or (C₃-C₆)cycloalkyl, or one is H and one is (C₁-C₆)alkyl or (C₃-C₆)cycloalkyl.

6. The method of claim 1 wherein 1 or 2 of R¹, R², R³ or R⁴ is (C₁-C₆)alkoxy.

7. The method of claim 1 wherein (R⁵)(R⁶)N— is in the para or 4-position.

8. The method of claim 1 wherein 1 or 2 of R¹, R², R³ and R⁴ is amino.

9. The method of claim 1 wherein R¹, R², R³ and R⁴ is H.

10. The method of claim 1 wherein the compound is procanamide, procaine, tetracaine, or lidocaine, or a pharmaceutically acceptable salt thereof.

11. The method of claim 1 wherein the compound is administered orally.

12. The method of claim 1 wherein the compound is administered parenterally.

13. The method of claim 1 wherein the compound is delivered by inhalation or insufflation.

14. The method of claim 1 wherein the neuropathological condition is Alzheimer's disease.

15. The method of claim 1 wherein the amount is effective to inhibit Aβ peptide-induced neurotoxicity.

16. The method of claim 15 wherein the amount is effective to inhibit Aβ₁₋₄₀, Aβ₁₋₄₂ or Aβ₁₋₄₃ neurotoxicity.

17. The method of claim 1 wherein the amount is effective to inhibit glutamate-induced neurotoxicity.

18. The method of claim 1 wherein the neuropathological condition is due to hyper-stimulation of a glumate pathway.

19. The method of claim 1 wherein the amount is effective to maintain ATP levels in neuronal cells.

20. The method of claim 1 wherein the compound of formula I or II is administered to a human.

21. The method of claim 20 wherein the human is in an early stage of AD

22. The method of claim 21 wherein the human is an AD patient.

23. The method of claim 20 wherein the human is afflicted with vascular dementia.

24. The method of claim 1 wherein R² is H.

25. The method of claim 24 wherein R³ is H.

26. The method of claim 1 wherein R⁴ is hydrogen.

27. The method of claim 25 wherein each of R¹, R² and R³ is H.

28. The method of claim 1 wherein the compound of formula (I) or (II) is administered in combination with a pharmaceutically acceptable carrier.

29. The method of claim 28 wherein the carrier is a liquid.

30. The method of claim 28 wherein the carrier is a solid.

31. A dosage form comprising a compound of formula (I) or (II) in combination with a pharmaceutically-acceptable carrier.

32. A therapeutic method to treat a neuropathy that involves glutamate network or pathway hyperactivity comprising administering to a mammal threatened with, or afflicted by, said neuropathy, an effective amount of a compound of formula I or formula II.


SEQUENCE LISTING (2 sequences)

SEQ ID NO: 1 - DNA, 24 bp, Artificial Sequence
  A synthetic primer
gactgtggtttgtgaagctgtcat

SEQ ID NO: 2 - DNA, 24 bp, Artificial Sequence
  A synthetic primer
aatacttctctcaccaccttggct